(19) United States
(12) Patent Application Publication
(10) Pub. No.: US 2009/0012488 A1
(43) Pub. Date: Jan. 8, 2009

(54) Super-Absorber Having Improved Smell-Inhibition

(75) Inventors: Volker Braig, Weinheim-Lutzelsachsen (DE); Thomas Servay, Worms (DE)

Correspondence Address: MARSHALL, GERSTEIN & BORUN LLP, 233 S. WACKER DRIVE, SUITE 6300, SEARS TOWER, CHICAGO, IL, 60606, US

(73) Assignee: BASF SE, Ludwigshafen (DE)

(21) Appl. No.: 12/281,545

(22) PCT Filed: Feb. 27, 2007
(86) PCT No.: PCT/EP07/51868

§ 371 Date: Sep. 3, 2008

(30) Foreign Application Priority Data
Mar. 10, 2006 (EP) 06110962.5

(51) Int. Cl. A61F 13/15; B01J 20/22; A61L 9/014; B01J 20/26

(52) U.S. Cl. 604/359; 502/401; 502/402; 424/76.1

(57) ABSTRACT

Unpleasant odors following absorption of bodily fluids in hygiene articles are controlled by utilizing in the hygiene articles a composition comprising at least one keto acid as well as a superabsorbent.

The present invention concerns superabsorbents with improved odor inhibition, processes for their production and their use.

Superabsorbents are known. Such materials are also commonly known by designations such as “high-swellability polymer”, “hydrogel” (often even used for the dry form), “hydrogel-forming polymer”, “water-absorbing polymer”, “absorbent gel-forming material”, “swellable resin”, “water-absorbing resin” or the like. The materials in question are crosslinked hydrophilic polymers, in particular polymers formed from (co)polymerized hydrophilic monomers, graft (co)polymers of one or more hydrophilic monomers on a suitable grafting base, crosslinked ethers of cellulose or starch, crosslinked carboxymethylcellulose, partially crosslinked polyalkylene oxide or natural products that are swellable in aqueous fluids, examples being guar derivatives, of which water-absorbing polymers based on partially neutralized acrylic acid are most widely used. The essential properties of superabsorbents are their ability to absorb and retain amounts of aqueous fluids equivalent to many times their own weight, even under moderate pressure. A superabsorbent which is used in the form of a dry powder transforms into a gel on taking up a liquid, specifically into a hydrogel when as usual taking up water. By far the most important field of use of superabsorbents is the absorbing of bodily fluids. Superabsorbents are used for example in diapers for infants, incontinence products for adults or feminine hygiene products. Examples of other fields of use are as water-retaining agents in market gardening, as water stores for protection against fire, for liquid absorption in food packaging or, in general, for absorbing moisture.

When superabsorbents are used in the hygiene sector, they are exposed to bodily fluids such as urine or menses. Such bodily fluids always contain malodorous components such as amines, fatty acids and other organic components which are responsible for unpleasant body odors. A further problem with such hygiene products is that the bodily fluids remain in the hygiene product for a certain time until the hygiene product is disposed of, and bacterial degradation of nitrogenous compounds present in the absorbed bodily fluids, an example being urea in urine, gives rise to ammonia or else other amines which likewise lead to a noticeable odor nuisance. Since correspondingly frequent changing of the hygiene product leads to an appreciable inconvenience and also cost for the user or his or her care persons, hygiene products where this odor nuisance is avoided are of advantage.

Various measures to avoid the odor nuisance are known. Odors can be masked by perfumization; the ammonia which results or amines can be removed by absorption or reaction, and the microbial degradation can be inhibited by means of biocides or urease inhibitors for example. These measures can be applied to the superabsorbent on the one hand and to the hygiene article on the other.

For instance, EP 1 358 894 A1 teaches hygiene articles which may include a series of odor-preventing additives, in particular anhydride groups, acid groups, cyclodextrins, biocides, surfactants having an HLB value of less than 11, absorbents such as zeolites, clay, activated carbon, silica or activated alumina, microorganisms which act as antagonists to undesirable odor-forming microorganisms, pH buffers or chelating agents. WO 03/076 514 A2 features a comprehensive overview of existing measures for avoiding unpleasant odors. The use of biocides such as bronopol or glyoxylic acid is disclosed among other measures. In addition, this reference teaches a superabsorbent containing anhydride groups capable of reacting with ammonia or amines and binding them in nonvolatile form as a result.

Ammonia and amines are bound at low pH as odor-neutral ammonium salts; in addition, a low pH inhibits the growth of ammonia-forming bacteria. EP 202 127 A2 accordingly teaches hygiene articles comprising a pH buffer such as an organic acid or else acid-modified cellulose, which keeps the pH of the skin in the range from 3.0 to 5.5. EP 316 518 A2 concerns superabsorbents formed from a partially neutralized polymeric organic acid which are optionally admixed with partially neutralized citric acid and which have a pH between 5.0 and 6.0 when in contact with liquid. WO 03/002 623 A1 teaches superabsorbents having a pH below 5.7. GB 2 296 013 A describes hygiene articles containing a polylactide layer, so that lactic acid is formed on contact with liquid, lowering the pH. WO 00/35 502 A1 teaches hygiene articles comprising a combination of a buffer having a pH in the range of 3.5-5.5 and lactic acid bacteria. WO 00/59 556 A2 utilizes a superabsorbent containing functional groups capable of reacting with ammonia or amines, in particular cyclic anhydrides, lactides or lactones of hydroxy acids. WO 01/32 226 A1 discloses a superabsorbent admixed with organic acids.

EP 894 502 A1 teaches the use of cyclodextrins as an absorbent for ammonia in hygiene articles. EP 1 034 800 A1 discloses hygiene articles which, as well as an absorbent for ammonia, in particular activated carbon, high surface area silica, clays, zeolites, diatomaceous earth, chitin, pH buffers, starch, cyclodextrin, or ion exchangers, also comprise oxidizing agents such as peracids or diacyl peroxides. WO 91/11 977 A1 relates to zeolites having an SiO2/Al2O3 ratio of less than 10 as absorbents for odors. WO 95/26 207 A1 utilizes zeolites having an average particle size of at least 200 micrometers for this purpose.

EP 1 149 597 A1 describes chitosan as an odor-inhibiting component for hygiene articles. EP 1 149 593 A1 teaches cationic polysaccharides, in particular chitin derivates or chitosan, in conjunction with a pH buffer which sets the pH in the range from 3.5 to 6.

EP 739 635 A1 teaches sodium metaborate and sodium tetraborate useful as urease inhibitors in superabsorbents. WO 94/25 077 A1 utilizes a mixture of alkali metal or alkaline earth metal tetraborate and boric acid, citric acid, tartaric acid or ascorbic acid as a buffer in pH range from 7 to 10. WO 03/053486 A1 discloses diapers comprising yucca palm extract as urease inhibitor. EP 1 214 878 A1 discloses the use of chelate complexes of bivalent metal ions such as of the copper complex of singly proteinated ethylenediaminetetraacetate as a urease inhibitor. WO 95/24173 A2 teaches the use of zeolites impregnated with bactericidal heavy metals such as silver, zinc or copper for odor control.

EP 311 344 A23 concerns hygiene articles which, as well as a pH buffer, comprise a biocide such as alkylammonium halides or bisguanidines. EP 389 023 A2 discloses hygiene articles comprising an odor control additive selected from biocides or absorbents, in particular molecular sieves. WO 98/26 808 A2 describes super-absorbents comprising cyclodextrins, zeolites, activated carbon, diatomaceous earth or acidic salt-forming substances as absorbents for odors and also biocides, urease inhibitors and pH regulators to inhibit odor formation.

WO 98/20 916 A1 utilizes a superabsorbent in hygiene articles which is coated with a biocide such as benzalkonium chloride or chlorhexidine. Prior provisional U.S. patent application of Jul. 27, 2005, U.S. Patent and Trademark Office Ser. No. 60/702,931, concerns storage-stable superabsorbents comprising substituted thiophosphoramides as odor inhibitor.

Mixing odor-absorbing high surface area materials in a sufficient amount with superabsorbents in the course of the production of hygiene articles lowers the fluid absorption capacity of the mixture. Superabsorbents having odor-inhibiting properties of themselves are those having a low pH. However, superabsorbents having a low pH are appreciably more difficult to produce than superabsorbents having a comparatively high pH. Less neutralized acrylic acid is slower to polymerize, and the acidic polymer, unlike the neutral or more basic polymer, tends to stick together, which massively impairs the necessary further processing (comminuting the gel, drying, grinding, classifying). In addition, acidic superabsorbents typically have poorer fluid retention under pressure. The use of biocides or antibiotics in hygiene articles, say as an addition to the superabsorbent, is disadvantageous, since these materials come into contact with the skin of the user by diffusion and in the process become active not only against odor-forming bacteria, but also in an unwanted manner. In addition, their use leads when the used hygiene articles are disposed of in the usual manner to an appreciable emission of biocides or antibiotics into the environment, impairing not only the functioning of water treatment plants, but also contributing to the formation of antibiotic-resistant bacterial strains. Similarly unwanted effects are associated with the use of bactericidal heavy metals such as zinc, silver or copper.

It is an object of the present invention to provide novel, improved or alternative superabsorbents or superabsorbent-containing compositions having odor-inhibiting properties or having improved odor inhibition. These shall moreover be stable in storage, more particularly neither discolor nor lose their odor-inhibiting properties in prolonged storage. Unwanted side effects on skin contact or emission of constituents into the environment should not arise. In addition, the superabsorbents or compositions shall have good liquid absorption and retention properties, of particular desirability being a rapid swelling capacity, good liquid-transporting properties in the gel bed coupled with high absorptive ability, high gel strength and good electrolyte tolerance. We have found that this object is achieved by compositions comprising superabsorbent and at least one keto acid.

When the composition of the present invention is used in a hygiene article it leads to unpleasant odors following contact with bodily fluids being avoided or at least reduced. It is believed that the incorporated keto acid removes ammonia by chemical reaction to form imide and/or ammonium salts, which leads to the observed odor-controlling effect. In addition, the keto acid lowers the pH in the superabsorbent bed in the hygiene article, so that bacterial growth is inhibited. The absorption and retention performance of the superabsorbent is not significantly impaired by the keto acid. It is not necessary, but possible, to use acidic superabsorbents. The keto acid does not impair stability in storage nor are undesirable effects on skin contact or emission into the environment observed or likely. The keto acid does not have a bactericidal effect.

Keto acids (short for the more correct “keto carboxylic acids”) are a subgroup of the oxo carboxylic acids, namely carboxylic acids which as well as a carboxyl group contain a ketone group, i.e., a group of the formula RR′C═O, where at least one of R and R′ bears a carboxyl function, but neither is a hydrogen atom. Aldehyde carboxylic acids are the further subgroup of the oxo carboxylic acids. In aldehyde carboxylic acids, one of R and R′ in the formula RR′C═O is hydrogen. The most simple representative of the aldehyde carboxylic acids is glyoxylic acid HCO—COOH, while the simplest representative of the keto carboxylic acids is pyruvic acid H₃C—CO—COOH.

The keto acid incorporated in the composition of the present invention has the general formula R¹—C(O)—R²—COOH, where R² may be omitted and preferably is. Preferably, the composition of the present invention thus comprises superabsorbent and at least one keto acid of the general formula R¹—CO—COOH, i.e., an alpha-keto acid or 2-oxo carboxylic acid.

R¹ is a linear, branched or cyclic organic radical which is optionally substituted. R¹ is for example a C₁- to C₃₀-alkyl radical, preferably a C₂- to C₁₀-alkyl radical, more preferably a C₃- to C₄₋alkyl radical. Examples of C₁- to C₁₀-alkyl radicals are methyl, ethyl, n-propyl, isopropyl, n-butyl, isobutyl, sec.-butyl, tert-butyl, n-pentyl, isopentyl, n-hexyl, isohexyl, n-heptyl, isoheptyl, n-octyl, isooctyl, n-nonyl, isononyl, n-decyl and isodecyl. n-Propyl and n-butyl are very particularly preferred alkyl. This alkyl radical is optionally substituted with one or more functional groups, in particular with one or more hydroxyl and/or carboxyl groups.

R², if present, is an organic group having two points of attachment, an example being a —(CH₂)ₙ— group, where n is generally from 1 to 4. The group may be linear, branched or cyclic and optionally substituted.

It is particularly preferable for the keto acid to be 2-oxo-L-gulonic acid (the L-enantiomer of 2-oxo-3,4,5,6-tetrahydroxyhexanoic acid) and/or 2-oxo-glutaric acid (2-oxo-pentane-1,5-dioic acid).

The preparation of keto acids is known. A common way to prepare alpha-keto acids is the oxidative deamination of alpha-amino acids. Many of these acids are common commercial products and are produced on a large industrial scale, including by ways other than oxidative deamination. For example, 2-oxo-L-gulonic acid is a direct precursor of vitamin C (the 2,3-enol form of 2-oxo-L-gulono-1,4-lactone) and is manufactured in very large volumes, usually fermentatively from sorbitol. Other alpha-keto acids such as pyruvic acid (2-oxopropanoic acid) or else beta-keto acids such as acetoacetic acid are likewise well-known commercial products.

We have found that keto acids, in particular alpha-keto acids, can be used to control unpleasant odors, in particular unpleasant odors due to ammonia.

The amount of keto acid in the composition of the present invention is generally at least 0.005% by weight, preferably at least 0.01% by weight, more preferably at least 0.1% by weight and most preferably at least 0.5% by weight and also generally not more than 15% by weight, preferably not more than 12% by weight and more preferably not more than 10% by weight, all based on the amount of superabsorbent in the composition. The amount of keto acid is for example 1% by weight, 2% by weight, 3% by weight, 4% by weight, 5% by weight, 6% by weight, 7% by weight, 8% by weight or 9% by weight, all based on the amount of superabsorbent in the composition. The composition, as well as keto acid and superabsorbent, may comprise further constituents, additives, auxiliaries or other components. It preferably consists essentially (i.e., except for inessential additives and/or auxiliaries) of superabsorbent and keto acid, or consists of superabsorbent and keto acid. The superabsorbent optionally comprises further additives or auxiliaries typically added to superabsorbents, examples being dustproofing agents, agents for improving the conveying properties or flowability of the superabsorbent.

The superabsorbent in the composition of the present invention is a customary superabsorbent capable of absorbing and retaining amounts of water equivalent to many times its own weight under a certain pressure. In general, it has a centrifugal retention capacity (CRC, method of measurement see hereinbelow) of at least 5 g/g, preferably at least 10 g/g and more preferably at least 15 g/g. Preferably, the superabsorbent is a crosslinked polymer based on partially neutralized acrylic acid, and more preferably it is surface postcrosslinked. A “superabsorbent” can also be a mixture of chemically different individual superabsorbents in that it is not so much the chemical composition which matters as the superabsorbing properties.

Processes for producing superabsorbents, including surface-postcrosslinked superabsorbents, are known. Synthetic superabsorbents are obtained for example by polymerization of a monomer solution comprising
a) at least one ethylenically unsaturated acid-functional monomer,
b) at least one crosslinker,
c) optionally one or more ethylenically and/or allylically unsaturated monomers copolymerizable with the monomer a), and
d) optionally one or more water-soluble polymers onto which the monomers a), b) and if appropriate c) can be at least partly grafted.

Suitable monomers a) are for example ethylenically unsaturated carboxylic acids, such as acrylic acid, methacrylic acid, maleic acid, fumaric acid and itaconic acid, or derivatives thereof, such as acrylamide, methacrylamide, acrylic esters and methacrylic esters. Acrylic acid and methacrylic acid are particularly preferred monomers. Acrylic acid is most preferable.

The monomers a) and especially acrylic acid comprise preferably up to 0.025% by weight of a hydroquinone half ether. Preferred hydroquinone half ethers are hydroquinone monomethyl ether (MEHQ) and/or tocopherols.

Tocopherol refers to compounds of the following formula:

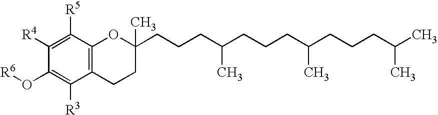

where R³ is hydrogen or methyl, R⁴ is hydrogen or methyl, R⁵ is hydrogen or methyl and R⁴ is hydrogen or an acid radical of 1 to 20 carbon atoms.

Preferred R⁶ radicals are acetyl, ascorbyl, succinyl, nicotinyl and other physiologically tolerable carboxylic acids. The carboxylic acids can be mono-, di- or tricarboxylic acids.

Preference is given to alpha-tocopherol where R³=R⁴=R⁵=methyl, especially racemic alpha-tocopherol. R⁶ is more preferably hydrogen or acetyl. RRR-alpha-Tocopherol is preferred in particular.

The monomer solution comprises preferably not more than 130 weight ppm, more preferably not more than 70 weight ppm, preferably not less than 10 weight ppm, more preferably not less than 30 weight ppm and especially about 50 weight ppm of hydroquinone half ether, all based on acrylic acid, with acrylic acid salts being arithmetically counted as acrylic acid. For example, the monomer solution can be produced using an acrylic acid having an appropriate hydroquinone half ether content.

Crosslinkers b) are compounds having at least two polymerizable groups which can be free-radically interpolymerized into the polymer network. Useful crosslinkers b) include for example ethylene glycol dimethacrylate, diethylene glycol diacrylate, allyl methacrylate, trimethylolpropane triacrylate, triallylamine, tetraallyloxyethane as described in EP 530 438 A1, di- and triacrylates as described in EP 547 847 A1, EP 559 476 A1, EP 632 068 A1, WO 93/21 237 A1, WO 03/104 299 A1, WO 03/104 300 A1, WO 03/104 301 A1 and DE 103 31 450 A1, mixed acrylates which, as well as acrylate groups, comprise further ethylenically unsaturated groups, as described in DE 103 31 456 A1 and WO 04/013 064 A2, or crosslinker mixtures as described for example in DE 195 43 368 A1, DE 196 46 484 A1, WO 90/15 830 A1 and WO 02/032962 A2.

Useful crosslinkers b) include in particular N,N′-methylenebisacrylamide and N,N′-methylenebismethacrylamide, esters of unsaturated mono- or polycarboxylic acids of polyols, such as diacrylate or triacrylate, for example butanediol diacrylate, butanediol dimethacrylate, ethylene glycol diacrylate, ethylene glycol dimethacrylate and also trimethylolpropane triacrylate and allyl compounds, such as allyl (meth)acrylate, triallyl cyanurate, diallyl maleate, polyallyl esters, tetraallyloxyethane, triallylamine, tetraallylethylenediamine, allyl esters of phosphoric acid and also vinylphosphonic acid derivatives as described for example in EP 343 427 A2. Useful crosslinkers b) further include pentaerythritol diallyl ether, pentaerythritol triallyl ether, pentaerythritol tetraallyl ether, polyethylene glycol diallyl ether, ethylene glycol diallyl ether, glycerol diallyl ether, glycerol triallyl ether, polyallyl ethers based on sorbitol, and also ethoxylated variants thereof. The process of the present invention may utilize di(meth)acrylates of polyethylene glycols, the polyethylene glycol used having a molecular weight between 300 and 1000.

However, particularly advantageous crosslinkers b) are di- and triacrylates of 3- to 15-tuply ethoxylated glycerol, of 3- to 15-tuply ethoxylated trimethylolpropane, of 3- to 15-tuply ethoxylated trimethylolethane, especially di- and triacrylates of 2- to 6-tuply ethoxylated glycerol or of 2- to 6-tuply ethoxylated trimethylolpropane, of 3-tuply propoxylated glycerol, of 3-tuply propoxylated trimethylolpropane, and also of 3-tuply mixedly ethoxylated or propoxylated glycerol, of 3-tuply mixedly ethoxylated or propoxylated trimethylolpropane, of 15-tuply ethoxylated glycerol, of 15-tuply ethoxylated trimethylolpropane, of 40-tuply ethoxylated glycerol, of 40-tuply ethoxylated trimethylolethane and also of 40-tuply ethoxylated trimethyloipropane.

Very particularly preferred for use as crosslinkers b) are diacrylated, dimethacrylated, triacrylated or trimethacrylated multiply ethoxylated and/or propoxylated glycerols as described for example in WO 03/104 301 A1. Di- and/or triacrylates of 3- to 10-tuply ethoxylated glycerol are particularly advantageous. Very particular preference is given to di- or triacrylates of 1- to 5-tuply ethoxylated and/or propoxylated glycerol. The triacrylates of 3- to 5-tuply ethoxylated and/or propoxylated glycerol are most preferred. These are notable for particularly low residual contents (typically below 10 weight ppm) in the water-absorbing polymer and the aqueous extracts of the water-absorbing polymers produced therewith have an almost unchanged surface tension (typically at least 0.068 N/m) compared with water at the same temperature.

Examples of ethylenically unsaturated monomers c) which are copolymerizable with the monomers a) are acrylamide, methacrylamide, crotonamide, dimethylaminoethyl methacrylate, dimethylaminoethyl acrylate, dimethylaminopropyl acrylate, diethylaminopropyl acrylate, dimethylaminobutyl acrylate, dimethylaminoethyl methacrylate, diethylaminoethyl methacrylate, dimethylaminoneopentyl acrylate and dimethylaminoneopentyl methacrylate.

Useful water-soluble polymers d) include polyvinyl alcohol, polyvinylpyrrolidone, starch, starch derivatives, polyglycols, polymers formally constructed wholly or partly of vinylamine monomers, such as partially or completely hydrolyzed polyvinylamide (so-called “polyvinylamine”) or polyacrylic acids, preferably polyvinyl alcohol and starch.

The polymerization is optionally carried out in the presence of customary polymerization regulators. Suitable polymerization regulators are for example thio compounds, such as thioglycolic acid, mercapto alcohols, for example 2-mercaptoethanol, mercaptopropanol and mercaptobutanol, dodecyl mercaptan, formic acid, ammonia and amines, for example ethanolamine, diethanolamine, triethanolamine, triethylamine, morpholine and piperidine.

The monomers (a) and (b) are (co)polymerized with each other, optionally in the presence of the comonomers (c) and/or the water-soluble polymers d), in 20% to 80%, preferably 20% to 50% and especially 30% to 45% by weight aqueous solution in the presence of polymerization initiators. Useful polymerization initiators include all compounds that disintegrate into free radicals under the polymerization conditions, examples being peroxides, hydroperoxides, hydrogen peroxide, persulfates, azo compounds and the so-called redox initiators. The use of water-soluble initiators is preferred. It is advantageous in some cases to use mixtures of various polymerization initiators, examples being mixtures of hydrogen peroxide and sodium or potassium peroxodisulfate. Mixtures of hydrogen peroxide and sodium peroxodisulfate can be used in any desired ratio. Suitable organic peroxides are for example acetylacetone peroxide, methyl ethyl ketone peroxide, tert-butyl hydroperoxide, cumene hydro-peroxide, tert-amyl perpivalate, tert-butyl perpivalate, tert-butyl perneohexanoate, tert-butyl perisobutyrate, tert-butyl per-2-ethylhexanoate, tert-butyl perisononanoate, tert-butyl permaleate, tert-butyl perbenzoate, tert-butyl per-3,5,5-trimethylhexanoate and tert-amyl perneodecanoate. Further suitable polymerization initiators are azo initiators, for example 2,2′-azobis(2-amidinopropane) dihydrochloride, 2,2′-azobis-(N,N-dimethylene)-isobutyramidine dihydrochloride, 2-(carbamoylazo)isobutyronitrile and 4,4′-azobis(4-cyanovaleric acid). The polymerization initiators mentioned are used in customary amounts, for example in amounts of from 0.01 to 5 mol %, preferably 0.1 to 2 mol %, based on the monomers to be polymerized.

The redox initiators comprise, as oxidizing component, at least one of the above-indicated per compounds and a reducing component, for example ascorbic acid, glucose, sorbose, ammonium bisulfite, ammonium sulfite, ammonium thiosulfate, ammonium hyposulfite, ammonium pyrosulfite, ammonium sulfide, alkali metal bisulfite, alkali metal sulfite, alkali metal thiosulfate, alkali metal hyposulfite, alkali metal pyrosulfite, alkali metal sulfide, metal salts, such as iron(II) ions or silver ions or sodium hydroxymethylsulfoxylate. The reducing component of the redox initiator is preferably ascorbic acid or sodium pyrosulfite. 1·10⁻⁵ to 1 mol % of the reducing component of the redox initiator and 1·10⁻⁵ to 5 mol % of the oxidizing component are used based on the amount of monomers used in the polymerization. Instead of the oxidizing component or in addition it is also possible to use one or more water-soluble azo initiators.

A redox initiator consisting of hydrogen peroxide, sodium peroxodisulfate and ascorbic acid is preferably used. These components are used for example in the concentrations of 1·10⁻² mol % of hydrogen peroxide, 0.084 mol % of sodium peroxodisulfate and 2.5·10⁻³ mol % of ascorbic acid, based on the monomers.

The aqueous monomer solution may comprise the initiator in dissolved or dispersed form. However, the initiators may also be added to the polymerization reactor separately from the monomer solution.

The preferred polymerization inhibitors require dissolved oxygen for optimum effect. Therefore, the polymerization inhibitors can be freed of dissolved oxygen prior to polymerization, by inertization, i.e., by flowing an inert gas, preferably nitrogen, through them. This is accomplished by means of inert gas, which can be introduced cocurrently, countercurrently or at entry angles in between. Good commixing can be achieved for example with nozzles, static or dynamic mixers or bubble columns. The oxygen content of the monomer solution is preferably lowered to less than 1 weight ppm and more preferably to less than 0.5 weight ppm prior to polymerization. The monomer solution is optionally passed through the reactor using an inert gas stream.

The preparation of a suitable polymer as well as further suitable hydrophilic ethylenically unsaturated monomers a) are described for example in DE 19941 423 A1, EP 686 650 A1, WO 01/45 758 A1 and WO 03/104 300 A1.

Superabsorbents are typically obtained by addition polymerization of an aqueous monomer solution and optionally a subsequent comminution of the hydrogel. Suitable methods of making are described in the literature. Superabsorbents are obtained for example by
gel polymerization in the batch process or tubular reactor and subsequent comminution in meat grinder, extruder or kneader, as described for example in EP 445 619 A2 and DE 19 846413 A1;
addition polymerization in kneader with continuous comminution by contrarotatory stirring shafts for example, as described for example in WO 01/38 402 A1;
addition polymerization on belt and subsequent comminution in meat grinder, extruder or kneader, as described for example in EP 955 086 A2, DE 3825 366 A1 or U.S. Pat. No. 6,241,928;
emulsion polymerization, which produces bead polymers having a relatively narrow gel size distribution, as described for example in EP 457 660 A1;
in situ addition polymerization of a woven fabric layer which, usually in a continuous operation, has previously been sprayed with aqueous monomer solution and subsequently been subjected to a photopolymerization, as described for example in WO 02/94 328 A2, WO 02/94 329 A1.

The cited references are expressly incorporated herein for details of process operation. The reaction is preferably carried out in a kneader or on a belt reactor.

Continuous gel polymerization is the economically preferred and therefore currently customary way of manufacturing superabsorbents. The process of continuous gel polymerization is carried out by first producing a monomer mixture by admixing the acrylic acid solution with the neutralizing agent, optional comonomers and/or further auxiliary materials at different times and/or locations and then transferring the mixture into the reactor or preparing the mixture as an initial charge in the reactor. The initiator system is added last to start the polymerization. The ensuing continuous process of polymerization involves a reaction to form a polymeric gel, i.e., a polymer swollen in the polymerization solvent—typically water—to form a gel, and the polymeric gel is already comminuted in the course of a stirred polymerization. The polymeric gel is subsequently dried, if necessary, and also chipped ground and sieved and is transferred for further surface treatment.

The acid groups of the hydrogels obtained have typically been partially neutralized, generally to an extent of at least 25 mol %, preferably to an extent of at least 27 mol % and more preferably at least 40 mol % and generally to an extent of not more than 85 mol %, preferably not more than 80 mol %, and more preferably not more than 75 mol %, for which the customary neutralizing agents can be used, preferably alkali metal hydroxides, alkali metal oxides, alkali metal carbonates or alkali metal bicarbonates and also mixtures thereof. Instead of alkali metal salts it is also possible to use ammonium salts. Sodium and potassium are particularly preferred as alkali metals, but most preference is given to sodium hydroxide, sodium carbonate or sodium bicarbonate and also mixtures thereof. Neutralization is customarily achieved by admixing the neutralizing agent as an aqueous solution or else preferably as a solid material. For example, sodium hydroxide having a water content of distinctly below 50% by weight can be present as a waxy mass having a melting point of above 23° C. In this case, metering as piecegoods or melt at elevated temperature is possible.

Neutralization can also be carried out after polymerization, at the hydrogel stage. But it is also possible to carry out the neutralization to the desired degree of neutralization wholly or partly prior to polymerization. In the case of partial neutralization and prior to polymerization, generally at least 10 mol %, preferably at least 15 mol % and also generally not more than 40 mol %, preferably not more than 30 mol % and more preferably not more than 25 mol % of the acid groups in the monomers used are neutralized prior to polymerization by adding a portion of the neutralizing agent to the monomer solution. The desired final degree of neutralization is in this case only set toward the end or after the polymerization, preferably at the level of the hydrogel prior to its drying. The monomer solution is neutralized by admixing the neutralizing agent. The hydrogel can be mechanically comminuted in the course of the neutralization, for example by means of a meat grinder or comparable apparatus for comminuting gellike masses, in which case the neutralizing agent can be sprayed, sprinkled or poured on and then carefully mixed in. To this end, the gel mass obtained can be repeatedly meat-grindered for homogenization.

Neutralization of the monomer solution to the desired final degree of neutralization prior to polymerization by addition of the neutralizing agent is preferred.

The as-polymerized gels are optionally maintained for some time, for example for at least 30 minutes, preferably at least 60 minutes and more preferably at least 90 minutes and also generally not more than 12 hours, preferably for not more than 8 hours and more preferably for not more than 6 hours at a temperature of generally at least 50° C. and preferably at least 70° C. and also generally not more than 130° C. and preferably not more than 100° C., which further improves their properties in many cases.

The neutralized hydrogel is then dried with a belt or drum dryer until the residual moisture content is preferably below 15% by weight and especially below 10% by weight, the water content being determined by EDANA (European Disposables and Nonwovens Association) recommended test method No. 430.2-02 “Moisture content”. The dry superabsorbent consequently contains up to 15% by weight of moisture and preferably not more than 10% by weight. The decisive criterion for classification as “dry” is in particular a sufficient flowability for handling as a powder, for example for pneumatic conveying, pack filling, sieving or other processing steps involved in solids processing technology. Optionally, however, drying can also be carried out using a fluidized bed dryer or a heated plowshare mixer. To obtain particularly colorless products, it is advantageous to dry this gel by ensuring rapid removal of the evaporating water. To this end, dryer temperature must be optimized, air feed and removal has to be policed, and at all times sufficient venting has to be ensured. Drying is naturally all the more simple—and the product all the more colorless—when the solids content of the gel is as high as possible. The solvent fraction at addition polymerization is therefore set such that the solid content of the gel prior to drying is therefore generally at least 20% by weight, preferably at least 25% by weight and more preferably at least 30% by weight and also generally not more than 90% by weight, preferably not more than 85% by weight and more preferably not more than 80% by weight. It is particularly advantageous to vent the dryer with nitrogen or some other nonoxidizing inert gas. Optionally, however, simply just the partial pressure of oxygen can be lowered during drying to prevent oxidative yellowing processes. But in general adequate venting and removal of the water vapor will likewise still lead to an acceptable product. A very short drying time is generally advantageous with regard to color and product quality.

The dried hydrogel (which is no longer a gel (even though often still called that) but a dry polymer having superabsorbing properties, which comes within the term “superabsorbent”) is preferably ground and sieved, useful grinding apparatus typically including roll mills, pin mills, hammer mills, cutting mills or swing mills. The particle size of the sieved, dry hydrogel is preferably below 1000 μm, more preferably below 900 μm and most preferably below 850 μm and preferably above 80 μm, more preferably above 90 μm and most preferably above 100 μm.

Very particular preference is given to a particle size (sieve cut) in the range from 106 to 850 μm. Particle size is determined according to EDANA (European Disposables and Nonwovens Association) recommended test method No. 420.2-02 “Particle size distribution”.

The dry superabsorbing polymers thus produced are typically known as “base polymers” and are then preferably surface postcrosslinked. Surface postcrosslinking can be accomplished in a conventional manner using dried, ground and classified polymeric particles. For surface postcrosslinking, compounds capable of reacting with the functional groups of the base polymer by crosslinking are applied, usually in the form of a solution, to the surface of the base polymer particles. Suitable postcrosslinkling agents are for example:
di- or polyepoxides, for example di- or polyglycidyl compounds such as diglycidyl phosphonate, ethylene glycol diglycidyl ether, bischlorohydrin ethers of polyalkylene glycols,
   alkoxysilyl compounds,
   polyaziridines, compounds comprising aziridine units and based on polyethers or substituted hydrocarbons, for example bis-N-aziridinomethane,
   polyamines or polyamidoamines and also their reaction products with epichlorohydrin,
   polyols such as ethylene glycol, 1,2-propanediol, 1,4-butanediol, glycerol, methyltriglycol, polyethylene glycols having an average molecular weight Mw of 200-10 000, di- and polyglycerol, pentaerythritol, sorbitol, the ethoxylates of these polyols and also their esters with carboxylic acids or carbonic acid such as ethylene carbonate or propylene carbonate,
   carbonic acid derivatives such as urea, thiourea, guanidine, dicyandiamide, 2-oxazolidinone and its derivatives, bisoxazoline, polyoxazolines, di- and polyisocyanates,
   di- and poly-N-methylol compounds such as for example methylenebis-(N-methylolmethacrylamide) or melamine-formaldehyde resins,
   compounds having two or more blocked isocyanate groups such as for example trimethylhexamethylene diisocyanate blocked with 2,2,3,6-tetramethylpiperidin-4-one.

If necessary, acidic catalysts can be added, examples being p-toluenesulfonic acid, phosphoric acid, boric acid or ammonium dihydrogenphosphate.

Particularly suitable postcrosslinking agents are di- or polyglycidyl compounds such as ethylene glycol diglycidyl ether, the reaction products of polyamidoamines with epichlorohydrin, 2-oxazolidinone and N-hydroxyethyl-2-oxazolidinone.

Surface postcrosslinking (often just “postcrosslinking”) is typically carried out by spraying a solution of the surface postcrosslinker (often just “postcrosslinker”) onto the hydrogel or the dry base polymer powder.

The solvent used for the surface postcrosslinker is a customary suitable solvent, examples being water, alcohols, DMF, DMSO and also mixtures thereof. Particular preference is given to water and water-alcohol mixtures, example being water-methanol, water-1,2-propanediol and water-1,3-propanediol.

The spraying with a solution of the postcrosslinker is preferably carried out in mixers having moving mixing implements, such as screw mixers, paddle mixers, disk mixers, plowshare mixers and shovel mixers. Particular preference is given to vertical mixers and very particular preference to plowshare mixers and shovel mixers. Useful and known mixers include for example Lödige®, Bepex®, Nauta®, Processall® and Schugi® mixers. Very particular preference is given to high speed mixers, for example of the Schugi-Flexomix® or Turbolizer® type.

The spraying with the crosslinker solution can be optionally followed by a thermal treatment step, essentially to effect the surface-postcrosslinking reaction (yet usually just referred to as “drying”), preferably in a downstream heated mixer (“dryer”) at a temperature of generally at least 50° C., preferably at least 80° C. and more preferably at least 90° C. and also generally not more than 250° C., preferably not more than 200° C. and more preferably not more than 150° C. The average residence time (i.e., the averaged residence time of the individual particles of superabsorbent) in the dryer of the superabsorbent to be treated is generally at least 1 minute, preferably at least 3 minutes and more preferably at least 5 minutes and also generally not more than 6 hours, preferably not more than 2 hours and more preferably not more than 1 hour. As well as the actual drying taking place, not only any products of scissioning present but also solvent fractions are removed. Thermal drying is carried out in customary dryers such as tray dryers, rotary tube ovens or heatable screws, preferably in contact dryers. Preference is given to the use of dryers in which the product is agitated, i.e., heated mixers, more preferably shovel dryers and most preferably disk dryers. Bepex® dryers and Nara® dryers are suitable dryers for example. Fluidized bed dryers can also be used. But drying can also take place in the mixer itself, by heating the jacket or blowing a preheated gas such as air into it. But it is also possible for example to utilize an azeotropic distillation as a drying process. The crosslinking reaction can take place not only before but also during drying.

A particularly preferred embodiment of the present invention additionally comprises modifying the hydrophilicity of the particle surface of the base polymers through formation of complexes. Complexes are formed on the outer shell of the particles by spraying with solutions of bi- or more highly valent cations, the cations being capable of reacting with the acid groups of the polymer to form complexes. Examples of bi- or more highly valent cations are polymers formally constructed wholly or partly of vinylamine monomers, such as partially or completely hydrolyzed polyvinylamide (so-called “polyvinylamine”) whose amine groups are always—even at very high pH values—partly present in a state of protonation to ammonium groups, or metal cations such as Mg²⁺, Ca²⁺, Al³⁺, Sc³⁺, Ti⁴⁺, Mn²⁺, Fe^{2+/3+}, Co²⁺, Ni²⁺, Cu²⁺, Zn²⁺, Y³⁺, Zr⁴⁺, La³⁺, Ce⁴⁺, Hf⁴⁺, and Au³⁺. Preferred metal cations are Mg²⁺, Ca²⁺, Al³⁺, Ti⁴⁺, Zr⁴⁺ and La³⁺, and particularly preferred metal cations are Al³⁺, Ti⁴⁺ and Zr⁴⁺. The metal cations can be used not only alone but also in admixture with each other. Of the metal cations mentioned, any metal salt can be used that has sufficient solubility in the solvent to be used. Metal salts with weakly complexing anions such as for example chloride, nitrate and sulfate, hydrogensulfate, carbonate, hydrogencarbonate, nitrate, phosphate, hydrogenphosphate, dihydrogenphosphate and carboxylate, such as acetate and lactate, are particularly suitable. It is particularly preferred to use aluminum sulfate. Useful solvents for the metal salts include water, alcohols, DMF, DMSO and also mixtures thereof. Particular preference is given to water and water-alcohol mixtures such as for example water-methanol, water-1,2-propanediol and water-1,3-propanediol.

The treatment of the base polymer with solution of a bi- or more highly valent cation is carried out in the same way as that with surface postcrosslinker, including the selective drying step. Surface postcrosslinker and polyvalent cation can be sprayed onto the base polymer in a conjoint solution or as separate solutions. The spraying of the metal salt solution onto the particles of superabsorbent can take place not only before but also after the surface-postcrosslinking operation. In a particularly preferred process, the spraying with the metal salt solution takes place in the same step as the spraying with the crosslinker solution, the two solutions being dispensed separately in succession or simultaneously through two nozzles, or crosslinker solution and metal salt solution can be conjointly sprayed through one nozzle.

When a drying step is carried out after surface postcrosslinking and/or treatment with complexing agent, it is advantageous but not absolutely necessary to cool the product after drying. Cooling can be carried out continuously or discontinuously, conveniently by conveying the product continuously into a cooler downstream of the dryer. Any apparatus known for removing heat from pulverulent solids can be used, in particular any apparatus mentioned above as a drying apparatus, provided it is supplied not with a heating medium but with a cooling medium such as for example with cooling water, so that heat is not introduced into the superabsorbent via the walls and, depending on the design, also via the stirrer elements or other heat-exchanging surfaces, but removed from the superabsorbent. Preference is given to the use of coolers in which the product is agitated, i.e., cooled mixers, for example shovel coolers, disk coolers or paddle coolers, for example Nara® or Bepex® coolers. The superabsorbent can also be cooled in a fluidized bed by blowing a cooled gas such as cold air into it. The cooling conditions are set such that a superabsorbent having the temperature desired for further processing is obtained. Typically, the average residence time in the cooler will be in general at least 1 minute, preferably at least 3 minutes and more preferably at least 5 minutes and also in general not more than 6 hours, preferably not more than 2 hours and more preferably not more than 1 hour, and cooling performance will be determined such that the product obtained has a temperature of generally at least 0° C., preferably at least 10° C. and more preferably at least 20° C. and also generally not more than 100° C., preferably not more than 80° C. and more preferably not more than 60° C.

Optionally, a further modification of the superabsorbent can be effected by admixing finely divided inorganic solids, for example silica, alumina, titania and iron(II) oxide, which further enhances the effects of the surface aftertreatment. It is particularly preferred to admix hydrophilic silica or an alumina having an average primary particle size in the range from 4 to 50 nm and a specific surface area of 50-450 m²/g. Finely divided organic solids are preferably admixed after the surface modification through crosslinking/complexing, but can also be carried out before or during these surface modifications.

Optionally, superabsorbent is provided with further customary additives and auxiliary materials to influence storage or handling properties. Examples thereof are colorations, opaque additions to improve the visibility of swollen gel, which is desirable in some applications, additions to improve the flowability of the powder, surfactants or the like. The superabsorbent is often admixed with dustproofing or dustbinding agents. Dustproofing or dustbinding agents are known in that for example polyether glycols such as polyethylene glycol having a molecular weight in the range from 400 to 20 000 g/mol, polyols such as glycerol, sorbitol, neopentylglycol or trimethylolpropane, which are optionally 7- to 20-tuply ethoxylated, are used. Similarly, a final water content can be set for the superabsorbent, if desired, by adding water.

The solids, additives and auxiliary materials can each be added in separate processing steps, but usually the most convenient method is to add them to the superabsorbent in the cooler, for example by spraying the superabsorbent with a solution or adding them in finely divided solid or in liquid form.

The surface-postcrosslinked superabsorbent is optionally ground and/or sieved in a conventional manner. Grinding is typically not necessary, but the sieving out of agglomerates which are formed or undersize is usually advisable to set the desired particle size distribution for the product. Agglomerates and undersize are either discarded or preferably returned into the process in a conventional manner and at a suitable point; agglomerates after comminution. The superabsorbent particle size is preferably not more than 1000 μm, more preferably not more than 900 μm, most preferably not more than 850 μm, and preferably at least 80 μm, more preferably at least 90 μm and most preferably at least 100 μm. Typical sieve cuts are for example 106 to 850 μm or 150 to 850 μm.

The composition of the present invention is produced by adding at least one keto acid to a superabsorbent. To this end, at least one of the following steps is carried out before, during or after the production of the superabsorbent:
i) mixing at least one keto acid with a superabsorbent;
ii) conjointly grinding the superabsorbent with at least one keto acid;
iii) spraying the superabsorbent with at least one keto acid, optionally dissolved in a solvent; and/or
iv) in the case of superabsorbents being produced by addition polymerization of at least one monomer, adding at least one keto acid to the monomer solution or to the reaction mixture during the addition polymerization.

To mix at least one keto acid with a superabsorbent, the keto acid or keto acid mixture is mixed with the superabsorbent (which may also be a base polymer prior to postcrosslinking) in any desired manner. Processes and apparatuses for mixing are known. The superabsorbent can for example be mixed with keto acid in the mixers and dryers mentioned above in relation to postcrosslinking, conveniently during postcrosslinking. However, subsequent admixing of the keto acid or acids is likewise possible, if these are present in a sufficiently firm form; mixing can also take place with cooling in the case of softer, for example waxy, substances.

The form of grinding involved in the conjoint grinding of at least one keto acid and a superabsorbent is not subject to any restriction. Suitable apparatuses are known and were described above in relation to the comminution of the base polymer. Conveniently, the keto acid or keto acid mixture is added during a grinding step in the production of the superabsorbent. In the case of softer, for example waxy, substances, the conjoint grinding can also take place with cooling.

When the keto acid or keto acids are applied by spraying, the form of spraying is likewise not subject to any restriction. The keto acid or keto acid mixture can be sprayed as a melt (preferably as a fine mist) or preferably in the form of a solution, for example and preferably during the postcrosslinking of the base polymers in the mixers mentioned in relation to the postcrosslinking of the base polymer and in which the surface postcrosslinker and/or the metal salt solution are sprayed onto the base polymer. The solvent used for the keto acid or keto acid mixture is a suitable solvent, for example water, water-acetone mixtures, water-propylene glycol mixtures or water-1,3-propanediol mixtures and also the solvents and solvent mixtures mentioned in relation to the postcrosslinking and metal salt treatment. The concentration of keto acid in the solution is generally at least 0.5% by weight, preferably at least 1% by weight and more preferably at least 2% by weight and also generally not more than 30% by weight, preferably not more than 20% by weight and more preferably not more than 10% by weight. Conveniently, the keto acid is applied together with surface-postcrosslinking agent and, if appropriate, metal salt in the surface-postcrosslinking step. Usually, the solutions are sprayed through separate nozzles, but if postcrosslinker, metal salt and keto acid do not enter into any undesirable reactions with one another they can also be sprayed in the form of a conjoint solution.

A further embodiment comprises using the above-described processes to produce a composition in accordance with the present invention that has a higher fraction of the at least one keto acid, generally at least 1% by weight, preferably at least 5% by weight and more preferably at least 10% by weight and also generally at most 50% by weight, preferably at most 40% by weight and more preferably at most 30% by weight. The composition thus obtained can then be diluted to the desired final keto acid content by admixing with further superabsorbent in customary apparatus.

If necessary to set the desired particle size distribution, the composition of the present invention is sieved once more following a subsequent application of or mixing with keto acid.

We have further found hygiene articles comprising the superabsorbent of the present invention. Hygiene articles in accordance with the present invention are for example those intended for use in mild or severe incontinence, such as for example inserts for severe or mild incontinence, incontinence briefs, also diapers, training pants for babies and infants or else feminine hygiene articles such as liners, sanitary napkins or tampons. Hygiene articles of this kind are known. The hygiene articles of the present invention differ from known hygiene articles in that they comprise the superabsorbent of the present invention. We have also found a process for producing hygiene articles, this process comprising utilizing at least one superabsorbent of the present invention in the manufacture of the hygiene article in question. Processes for producing hygiene articles using superabsorbent are otherwise known.

The present invention further provides for the use of the composition of the present invention in training pants for children, shoe inserts and other hygiene articles to absorb bodily fluids. The composition of the present invention can also be used in other technical and industrial fields where liquids, in particular water or aqueous solutions, are absorbed. These fields are for example storage, packaging, transportation (as constituents of packaging material for water- or moisture-sensitive articles, for example for flower transportation, also as protection against mechanical impacts); animal hygiene (in cat litter); food packaging (transportation of fish, fresh meat; absorption of water, blood in fresh fish or meat packs); medicine (wound plasters, water-absorbing material for burn dressings or for other weeping wounds), cosmetics (carrier material for pharmachemicals and medicaments, rheumatic plasters, ultrasonic gel, cooling gel, cosmetic thickeners, sun protection); thickeners for oil-in-water and water-in-oil emulsions; textiles (moisture regulation in textiles, shoe inserts, for evaporative cooling, for example in protective clothing, gloves, headbands); chemical engineering applications (as a catalyst for organic reactions, to immobilize large functional molecules such as enzymes, as adhesion agent in relation to agglomerations, heat storage media, filter aids, hydrophilic component in polymeric laminates, dispersants, superplasticizers); as auxiliaries in powder injection molding, in building construction and engineering (installation, in loam-based renders, as a vibration-inhibiting medium, auxiliaries in tunnel excavations in water-rich ground, cable sheathing); water treatment, waste treatment, water removal (deicing agents, reusable sandbags); cleaning; agritech (irrigation, retention of melt water and dew deposits, composting additive, protection of forests against fungal/insect infestation, delayed release of active components to plants); for firefighting or for fire protection; coextrusion agents in thermoplastic polymers (for example to hydrophilcize multilayered films); production of films and thermoplastic moldings able to absorb water (for example rain and dew water storage films for agriculture; superabsorbent-containing films for keeping fruit and vegetables fresh which are packed in moist films; superabsorbent-polystyrene coextrudates, for example for food packaging such as meat, fish, poultry, fruit and vegetables); or as carrier substance in formulations of active components (pharma, crop protection).

### Test Methods

### Centrifuge Retention Capacity (CRC):

Centrifuge retention capacity (CRC) is determined by EDANA (European Disposables and Nonwovens Association, Avenue Eugene Plasky 157, 1030 Brussels, Belgium) recommended test method No. 441.2-02 “Centrifuge Retention Capacity”, which is available from EDANA at the address given.

### Determination of Odor Inhibition:

To evaluate the odor-inhibiting effect of the compositions of the present invention, the inhibition of the activity of urease in the formation of ammonia from urea is determined. The determination is carried out at room temperature. 2 g of the substance to be tested (superabsorbent, composition according to the present invention) are weighed into a 100 ml Erlenmeyer flask. 30 mg of solid urease (from jack beans; lyophilized 5 U/mg for urea assay in serum; Merck, article No. 4194753) is weighed into a 100 ml glass beaker. 50 ml of 0.9% sodium chloride solution in urea (8.56 g/L) are added to the urease. The entire contents of the glass beaker are poured swiftly onto the sample in the Erlenmeyer flask, a diffusion tubelet (Dräger Röhrchen, ammonia 20/a-D, 20-1500 ppm*h, order No. 8101301) is as intended broken open, inserted into a rubber stopper which fits the Erlenmeyer flask and the Erlenmeyer flask is sealed with the stopper such that the open end of the diffusion tubelet points inward. The value displayed by the diffusion tubelet is read off every 30 minutes and recorded for a total of 6 hours. The measurement is in each case carried out as a duplicate determination, and the result reported is the average between the two readings which has been rounded to the nearest whole number.

### EXAMPLES

The surface-postcrosslinked superabsorbent used in the examples which follow was produced by the process of Example 1 of WO 01/038 402 A1, except with a degree of neutralization of 72 mol % (instead of 77 mol %) and in a sieve cut of 106 to 850 μm (instead of <800 μm). Weight percentages reported for the keto acids used are based on the amount of superabsorbent used.

### Comparative Example V1

Superabsorbent was tested without added keto acid.

### Comparative Example V2

Superabsorbent was mixed in a commercially available kitchen machine with 1.5% by weight of glyoxylic acid by spraying with the appropriate amount of 50% by weight aqueous glyoxylic acid solution while stirring.

### Comparative Example V3

Superabsorbent was mixed in a commercially available kitchen machine with 3.0% by weight of glyoxylic acid by spraying with the appropriate amount of 50% by weight aqueous glyoxylic acid solution while stirring.

### Comparative Example V4

Superabsorbent was mixed in a commercially available kitchen machine with 6.0% by weight of glyoxylic acid by spraying with the appropriate amount of 50% by weight aqueous glyoxylic acid solution while stirring.

### Example 1

Superabsorbent was mixed with 0.5% by weight of finely ground 2-keto-L-gulonic acid by tumbling in a tumble mixer for about 20 minutes.

### Example 2

Superabsorbent was mixed with 1.0% by weight of finely ground 2-keto-L-gulonic acid by tumbling in a tumble mixer for about 20 minutes.

### Example 3

Superabsorbent was mixed with 3.0% by weight of finely ground 2-keto-L-gulonic acid by tumbling in a tumble mixer for about 20 minutes.

### Example 4

Superabsorbent was mixed with 0.5% by weight of finely ground 2-oxoglutaric acid by tumbling in a tumble mixer for about 20 minutes.

### Example 5

Superabsorbent was mixed with 1.0% by weight of finely ground 2-oxoglutaric acid by tumbling in a tumble mixer for about 20 minutes.

### Example 6

Superabsorbent was mixed with 3.0% by weight of finely ground 2-oxoglutaric acid by tumbling in a tumble mixer for about 20 minutes.

CRC and the odor-inhibiting effect of the superabsorbents and compositions obtained in the examples was determined as described above. The results are listed in table 1.

Comparing the inventive with the comparative examples shows that, although the known odor inhibitor glyoxylic acid demonstrates an odor-inhibiting effect, the keto acids have a far stronger odor-inhibiting effect in a far lower concentration. The absorptive performance of the superabsorbent is only insignificantly impaired by the added acid.

TABLE 1

| | Example No. | | | | | | | | | |
|---|---|---|---|---|---|---|---|---|---|---|
| | V1 | V2 | V3 | V4 | 1 | 2 | 3 | 4 | 5 | 6 |
| | — | Glyoxylic acid [% by weight] | | | 2-Keto-L-gulonic acid, in % by weight | | | 2-Oxoglutaric acid, in % by weight | | |
| | — | 1.5 | 3.0 | 6.0 | 0.5 | 1.0 | 3.0 | 0.5 | 3.0 | 1.0 |
| | CRC [g/g] | | | | | | | | | |
| | 30.0 | 28.5 | 28.3 | 27.3 | 29.6 | 28.9 | 28.4 | 28.9 | 27.7 | 29.8 |
| Time [h] | Determination of odor inhibition | | | | | | | | | |
| 0.5 | 5 | 35 | 45 | 48 | 10 | 3 | 5 | 3 | 5 | 3 |
| 1 | 16.5 | 55 | 78 | 85 | 20 | 3 | 5 | 15 | 13 | 3 |
| 1.5 | 45 | 83 | 100 | 100 | 55 | 5 | 8 | 40 | 15 | 3 |
| 2 | 100 | 100 | 130 | 115 | 100 | 8 | 8 | 85 | 25 | 3 |
| 2.5 | 185 | 125 | 145 | 135 | 160 | 10 | 8 | 170 | 28 | 3 |
| 3 | 260 | 160 | 180 | 150 | 205 | 13 | 8 | 255 | 30 | 3 |
| 3.5 | 350 | 215 | 225 | 180 | 270 | 23 | 8 | 310 | 33 | 3 |
| 4 | 440 | 250 | 275 | 200 | 325 | 30 | 8 | 410 | 33 | 3 |
| 4.5 | 525 | 345 | 350 | 205 | 405 | 55 | 8 | 495 | 33 | 3 |
| 5 | 625 | 400 | 400 | 235 | 425 | 75 | 8 | 590 | 33 | 3 |
| 5.5 | 740 | 450 | 450 | 270 | 500 | 105 | 8 | 675 | 33 | 3 |
| 6 | 850 | 550 | 575 | 300 | 575 | 138 | 8 | 800 | 33 | 3 |

### Examples 7-12

In each case, 1.0 g of superabsorbent according to Examples 2 (1.0% by weight of keto-L-gulonic acid) and 3 (3.0% by weight of keto-L-gulonic acid) was suspended in 100 ml of physiological saline (0.9% by weight NaCl solution in water) and admixed in each case with 0.1 ml of a germ suspension. Following incubation times at room temperature of 10 minutes, 4 hours and 8 hours, samples were taken and the number of colony-forming units (cfu) was determined. The following three germ suspensions having the stated starting concentrations were used:

| | |
|---|---|
| Escherichia coli | 1.6 · 10⁶ cfu/ml |
| Staphylococcus aureus | 2.2 · 10⁶ cfu/ml |
| Proteus mirabilis | 1.4 · 10⁶ cfu/ml |

The results are summarized below in table 2:

TABLE 2

| | | | Germ count [in | | |
|---|---|---|---|---|---|
| | | Super- | 10⁵ cfu/g] after | | |
| Example | Germ | absorbent | 10 min | 4 h | 8 h |
| 7 | Escherichia coli | Ex. 2 | 1.3 | 2.4 | 2.9 |
| 8 | Staphylococcus aureus | Ex. 2 | 1.7 | 2.5 | 2.7 |
| 9 | Proteus mirabilis | Ex. 2 | 1.9 | 2.9 | 3.0 |
| 10 | Escherichia coli | Ex. 3 | 2.3 | 1.2 | 2.4 |
| 11 | Staphylococcus aureus | Ex. 3 | 1.5 | 1.9 | 1.4 |
| 12 | Proteus mirabilis | Ex. 3 | 1.8 | 1.7 | 3.1 |

Examples 7-12 show that the superabsorbent comprising keto-L-gulonic acid has no significant bactericidal effect.

1. A composition comprising a superabsorbent and at least one keto acid.

2. The composition according to claim 1 which comprises at least one alpha-keto acid.

3. The composition according to claim 2 which comprises 2-keto-L-gulonic acid, 2-ketoglutaric acid, or a mixture thereof.

4. The composition according to claim 1 wherein the amount of keto acid is in the range from 0.005% to 15% by weight, based on the amount of superabsorbent.

5. The composition according to claim 1 wherein the superabsorbent is a crosslinked polymer based on partially neutralized acrylic acid.

6. The composition according to claim 1 wherein the superabsorbent is surface postcrosslinked.

7. A process for producing a composition of claim 1, which comprises producing a superabsorbent by performing at least one of the following steps:
i) admixing at least one keto acid;
ii) conjointly grinding the superabsorbent with at least one keto acid;
iii) spraying the superabsorbent with at least one keto acid, optionally dissolved in a solvent; and/or
iv) in the case of the superabsorbent being produced by addition polymerization of at least one monomer, adding at least one keto acid to a monomer solution or to a reaction mixture during the addition polymerization.

8. A hygiene article comprising a composition of claim 1.

9. The hygiene article according to claim 8 for use in severe incontinence and in mild incontinence.

10. (canceled)

11. (canceled)

12. The method according to claim 14 wherein said unpleasant odor is due to ammonia.

13. The method according to claim 14 wherein an alpha-keto acid is used.

14. A method of controlling an unpleasant odor comprising use of alpha-keto acid.
